(19) United States
(12) Patent Application Publication
(10) Pub. No.: US 2023/0172473 A1
(43) Pub. Date: Jun. 8, 2023

(54) WEARABLE BIO-ELECTROMAGNETIC SENSOR AND METHOD OF MEASURING PHYSIOLOGICAL PARAMETERS OF A BODY TISSUE

(71) Applicant: TALLINN UNIVERSITY OF TECHNOLOGY, Tallinn (EE)

(72) Inventors: Jaan OJARAND, Tallinn (EE); Mart MIN, Tallinn (EE); Olev MÄRTENS, Tallinn (EE); Raul LAND, Tallinn (EE); Eiko PRIIDEL, Tallinn (EE); Paul ANNUS, Tallinn (EE)

(21) Appl. No.: 17/927,402

(22) PCT Filed: May 25, 2021
(86) PCT No.: PCT/IB2021/054556

§ 371 Date: Nov. 23, 2022

Related U.S. Application Data
(60) Provisional application No. 63/029,581, filed on May 25, 2020.

(30) Foreign Application Priority Data
May 17, 2021 (EE) P202100015

(51) Int. Cl. A61B 5/05; A61B 5/08; A61B 5/02; A61B 5/00

(52) U.S. Cl. CPC A61B 5/05; A61B 5/08; A61B 5/02; A61B 5/6804; A61B 5/6831

(57) ABSTRACT

A wearable bio-electromagnetic sensor comprises an electronic unit containing a means for generating electrical current, and an electromagnetic interface for transforming the generated electrical current into an electromagnetic field applied to a vascularized body tissue. Next, the wearable bio-electromagnetic sensor contains a means for analog signal processing an electrical response of cardiopulmonary system to the applied electromagnetic field. After analog processing of said electrical response, a digital post-processing of digitized electrical response takes place in a means for digital signal processing, embedded into said electronic unit of the wearable bio-electromagnetic sensor. As a result of analog and digital signal processing, an information is extracted, which makes possible medical diagnosing of both, pulmonary and cardiovascular system, separately or simultaneously. The used work principle is following: the applied electromagnetic field induces electrical current inside the body tissue, electrical impedance to which changes correspondingly to breathing and heart beating. Said electrical impedance of varies during every breathing cycle correspondingly to oxygen transporting through arteries and oxygen uptake by capillaries, also due to biomechanical enlargement and narrowing of arteries correspondingly to blood pressure variations.

### FIELD OF INVENTION

The invention relates to personal medical devices, more specifically to wearable bioelectromagnetic sensor devices.

### BACKGROUND ART

Electrical impedance characterizes the properties of different materials, structures and processes as composition of metals, structures of materials, electro-chemical reactions as corrosion, etc. [1]. Electrical bio-impedance (EBI) is the electrical impedance of biological matter, describing living biological materials (cells, tissues, organs) and such the physiological processes as breathing, heart beating, flowing of blood and tissue oxygenation. In summary, electrical bio-impedance allows to measure and analyze the cardiopulmonary and vascular dynamics, which are the most necessary physiological processes for medical diagnosing of human health [2]. To avoid serious electrode problems and reduce artefacts, the non-contact sensing methods are of interest [3] by using both capacitive and inductive coupling.

An important application of the impedance is monitoring of the hemodynamics of the person. One specific application could be impedance cardiography (ICG [4]), but more generally, monitoring of cardiac and respiratory data [5].

[1] Y. Barsukov and J. Macdonald, Impedance Spectroscopy: Theory, Experiment, and Applications, 2nd Edn., January 2005

[2] S. Grimnes and O. Martinsen, Bioimpedance and Bioelectricity Basics, January 2008.

[3] H. Sanier, S. E. J. Knobel, N. Shuetz, and T. Nef, “Contact-free signals as a new digital biomarker for cardiovascular disease: chances and challenges,” European Heart Journal—Digital Health, vol. 1, no. 1, pp. 30-39, 2020.

[4] W. Kubicek, R. Patterson, and D. Witsoe, “Impedance cardiography as a noninvasive method of monitoring cardiac function and other parameters of the cardiovascular system,” Annals of the New York Academy of Sciences, vol. 170, pp. 724-732, December 2006, First Published 1970.

[5] D. Naranjo, J. Reina-Tosina, and M. Min, “Fundamentals, recent advances, and future challenges in bioimpedance devices for healthcare applications,” Journal of Sensors, vol. 2019, pp. 1-42, July 2019.

US2016/0089053 discloses a noninvasive method and apparatus for determination of heart rate, heart stroke volume, and cardiac output from thoracic bioimpedance signals and electrocardiograms. The electrodes are attached to the forehead, neck and chest area of the patient.

US20100076328 discloses a pulse wave measurement electrode unit in the form of a cuff with two current electrodes and two voltage electrodes to acquire a volume pulse wave of an artery by measuring a fluctuation of a biological impedance, and a pulse wave measurement device equipped with the same.

U.S. Pat. No. 9,161,699 discloses a device for the non-invasive determination of arterial blood pressure of a human or animal body, comprising at least a bioimpedance measuring device having a plurality of electrode pairs for capturing the admittance signals caused by an impressed alternating current on at least one first section of the body, wherein the captured admittance signals correspond to a composite signal made of signal components of a pulse admittance, a respiration admittance as well as a base admittance, including also at least one device for the non-invasive measurement of the blood pressure. The device can be attached to the arm of a person.

In known devices, usually a galvanic (ohmic) contact is used. Such devices cannot be used for continuing measurements and monitoring since such contact is very hard to maintain when the object such as the arm or wrist is moving. A device is needed that is not sensitive to movement.

Impedance of the chest and head can be measured not only by electrically conductive electrodes placed on the body, but also by using inductive (magnetic induction) coupling [6], enabling not only cardiovascular but also respiratory monitoring [7].

[6] P. P. Tarjan and R. McFee, “Electrodeless measurements of the effective resistivity of the human torso and head by magnetic induction,” IEEE Transactions on Biomedical Engineering, vol. BME-15, no. 4, pp. 266-278, 1968.

[7] D. Teichmann, J. Foussier, and S. Leonhardt, “Respiration monitoring based on magnetic induction using a single coil,” in 2010 Biomedical Circuits and Systems Conference (BioCAS), 2010, pp. 37-40.

However, such devices use either solenoid or planar coil. Neither coils can generate magnetic field inside the body or body member that is directed along the body or body member, such as blood vessel or another organ.

### SUMMARY OF THE INVENTION

One aspect of the invention is a bio-electromagnetic sensor device, comprising a means for generating an electrical current, and an electromagnetic interface for transforming the electrical current into an electromagnetic field to induce alternating current within a portion of the body with a convex surface by directing said current through a cross-section of said portion of the body part (arm, neck, head, chest, foot, waist, etc.), a toroidal magnet is introduced a core shape of which follows said convex surface.

The shape of the core follows this convex surface to a full or incomplete but appreciable extent, for example, a half, quarter or tenths of the surface.

The convex surface is a closed surface—tubular, either with a circular cross-section (classic tube) or corresponding to its distorted variant (eg a blood vessel, arm or leg). For example, an ellipse or any shape with a closed surface line in which a convex surface part is distinguishable.

Another aspect of the invention, a wearable bio-electromagnetic sensor comprises an electronic unit comprising a means for generating electrical current, and an electromagnetic interface for transforming the generated electrical current into an electromagnetic field applied to a body tissue, such as vascularized body tissue. The wearable bio-electromagnetic sensor further comprises a means for analog signal processing of an electrical response of the cardiopulmonary system caused by the applied electromagnetic field. After analog processing of said electrical response, a digital post-processing of digitized electrical response takes place in a means for digital signal processing, embedded into said electronic unit of the wearable bio-electromagnetic sensor. The principle is the following: the applied electromagnetic field induces electrical current inside the body tissue, e.g., in a blood vessel, the electrical impedance to which changes correspondingly to changes in blood flow, such changes representing breathing and heart beating. Said electrical impedance varies during every breathing cycle correspondingly to oxygen transporting through arteries and oxygen uptake by capillaries, also due to biomechanical enlargement and narrowing of arteries correspondingly to blood pressure variations.

As a result of analog and digital signal processing, an information is extracted, which makes it possible to determine the blood pressure, blood pressure variations, heart rate, blood pressure waveforms, blood oxygen content and other parameters of the hemodynamics of a person.

Such parameters could be also used in medical diagnosing of both pulmonary and cardiovascular system, separately or simultaneously.

### SHORT DESCRIPTION OF FIGURES

The invention is now described with reference to enclosed illustrative drawings and photographs.

FIG. 1 is a sensor device according to one embodiment of the invention;

FIG. 2 is a sensor device according to another embodiment of the invention;

FIG. 3 is a photo of a sensor device according to another embodiment of the invention;

FIGS. 4A and 4B are photos of a sensor device according to other embodiments of the invention;

FIG. 5 is a photo of a sensor device according to another embodiment of the invention;

FIG. 6 is a photo showing the sensor device as shown in FIGS. 3 to 5, strapped to the wrist of a person;

FIG. 7A shows the shape and direction of the magnetic field and the direction of the induced current when a toroidal core is placed on the wrist and 7B shows the creation of a magnetic field in toroidal core, and the current induced by it in a conductive material, e.g. in a blood vessel;

FIG. 8 is an equivalent scheme for connecting a sensor device with a body tissue according to one aspect of the invention;

FIG. 9 is an equivalent scheme for connecting a sensor device with a body tissue according to another aspect of the invention.;

FIG. 10 is a principal measurement scheme according to aspects of the invention.

FIG. 11 is a principal measurement scheme for measuring impedance Z of a parallel LRC circuit.

FIG. 12 is a graph showing frequency response of magnitude (upper graph) and phase (lower graph) of the impedance Z for the circuit of FIG. 11.

FIG. 13 is a graph showing the impact of the variation of capacitance C of the body tissue.

FIG. 14 is a graph showing the impact of the variation of losses in body tissue

FIG. 15 shows the changes of the resonant frequency between 4.86 and 4.94 MHz of parallel RLC circuit

FIG. 16 shows a graph of a measured impedance signal, phase modulated due to breathing.

FIG. 17 shows a graph of measured impedance signal, phase modulated due to heart beating.

FIG. 18 shows a graph of measured impedance signal, level modulated due to breathing.

FIG. 19 shows a graph of measured impedance signal, level modulated due to heart beating.

FIG. 20 shows changes of the resonant frequency Δf (upper graph) and phase Δφ (lower graph) of parallel RLC circuit (see FIG. 11) due to variation of the body tissue 1 capacitance 11 between 31.5 and 32.5 pF.

FIG. 21 shows changes of the level at resonant frequency of parallel RLC circuit (see FIG. 11) due to variation of losses in the body tissue 1, the loss resistance Rl (12) changes between 45 and 55 kOhm

FIG. 22 shows an alternative measurement scheme.

FIG. 23 shows another alternative measurement scheme.

FIG. 24A to D show four alternative ways of circuit closures according to the invention.

FIG. 25 is a lung respiration curve with cardiac pulsation on it as obtained by the measurement schemes of FIGS. 24A to 24D.

FIG. 26A shows a toroidal core sensor placed on the wrist with a measuring device, using an electrode placed on both sides of the sensor to close the current flow path. FIG. 26B is a photograph of an experimental design of the sensor circuit of FIG. 26A.

FIG. 27 shows a measured heart work curve with a slow change in amplitude due to respiration.

### DETAILED DESCRIPTION OF THE INVENTION

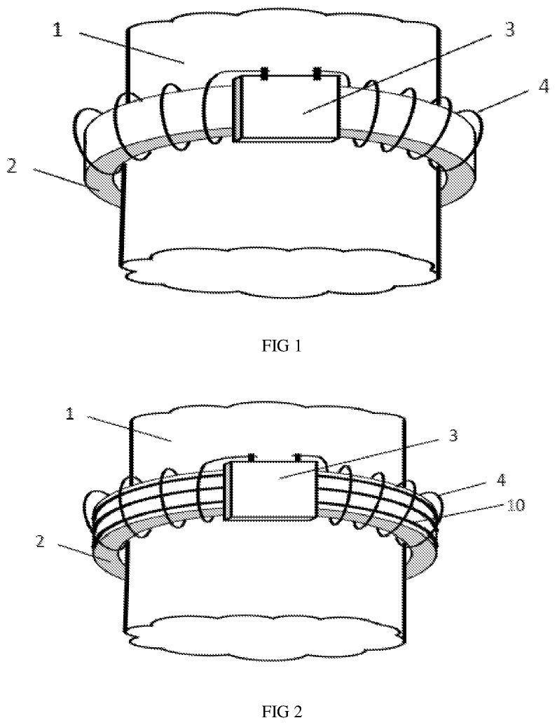

FIG. 1 shows a sensor device according to one embodiment of the invention. A round shape body part 1 (wrist, finger, arm, hand, leg, chest, neck, head, etc.) is surrounded by a circular strip 2, around of which a spiral winding 4 is wound, forming together a toroidal magnetic coil, to which an electronic unit 3 is attached. The magnetic coil (shown as 2 and 4) interacts with body 1 via electromagnetic field (galvanic contact is absent), forming an electromagnetic interface 5 (see FIGS. 8 to 11) for transforming electrical current from electronic unit 3 into an electromagnetic field applied to the body part 1. The electronic unit 3 contains a means for generating electrical current 6 (see FIGS. 8 to 11) into the spiral winding 4, a means for analog signal processing 7 (see FIGS. 8 to 11), a means for digital signal processing 8 (see FIG. 10), a means for digital communications 9 (see FIG. 10) and other electronic circuits supporting the work of electronic unit 3.

The acquired waveform of breathing satisfies the best expectations, but heart-beating response in composite waveform is relatively low and contains disturbances.

To overcome the problems, a sensor shown in FIGS. 26A and 26B is preferable. The sensor is supplemented with two capacitive electrodes 13 and 14 on each side of the circular strip, the two capacitive electrodes connected with each other directly, e.g., through wire connection 15, not via body. This enables to measure the blood pulsation in the wrist area only, not including other parts of the body. Moreover, the capacitances of supplemented electrodes and inductance of the coil were tuned to have a serial resonance at the frequency of 10 MHz to measure the loss resistance directly.

FIG. 2 shows a sensor device according to another embodiment of the invention, a complemented version of the bio-electromagnetic sensor. Similarly to the version in FIG. 1, the round shape body part 1 (hand, wrist finger, arm, leg, chest, neck, head, etc.) is surrounded by a strip 2, to which the electronic unit 3 is attached. 5 The magnetic coil is formed from the spiral winding 4 wound around the strip 2, to which a solenoidal winding 10 has been wound (e.g. three or more windings, as shown in FIG. 2). The magnetic fields created by windings 4 and 5, are perpendicular and used for focusing the magnetic induction into the required body region.

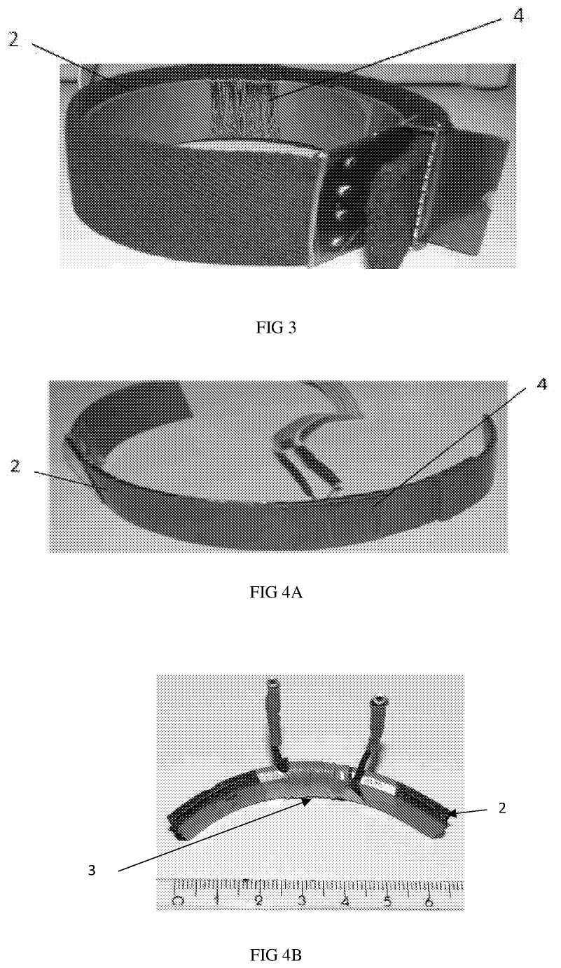

FIG. 3 is a photo of a sensor device according to yet another embodiment of the sensor device, comprising a closed strip 2, around of which the winding 4 from an insulated electric wire is wounded as in FIG. 1. The strip 2 is constructed on the bases of flexible magnetic material.

FIGS. 4A and 4B show sensor devices according to yet another embodiment of the sensor device with open magnetic flux circuit, having an interrupted magnetic strip 2 with a relatively short winding 4 on it. Such interrupted magnetic strip 2 can be as short as ½ to 1/10 of the full extent of the toroid.

FIG. 5 is a photo of a sensor device, having a strip 2, to which an electronic unit 3 is attached, has a spiral winding 4 around the strip 2 connected electrically with the electronic unit 3. The strip 2 has also the solenoidal winding 10 (see FIG. 2) under the coil with spiral winding 4.

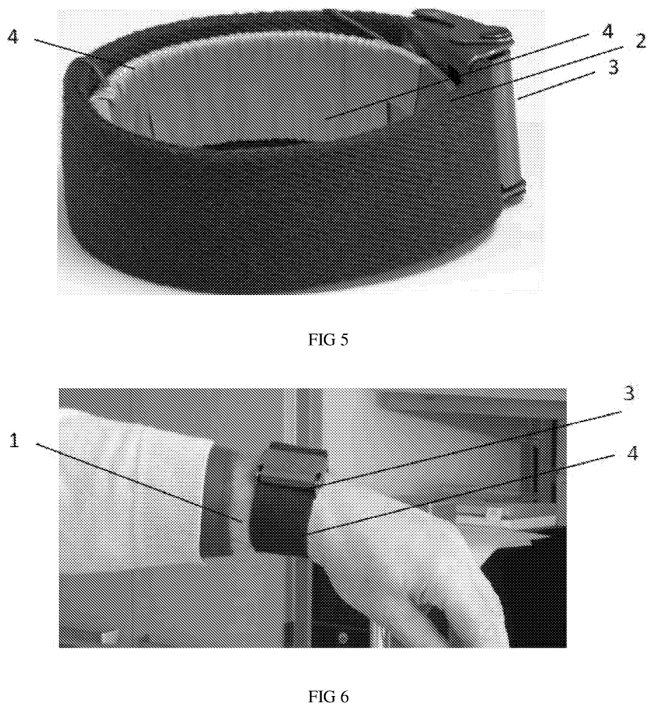

FIG. 6 is a photo of a sensor device, where a wearable bio-electromagnetic sensor, e.g., as shown of FIGS. 1 to 5, is strapped to the wrist (the round shape body part 1), where 3 is the electronic unit, 5 and 4 is the coil.

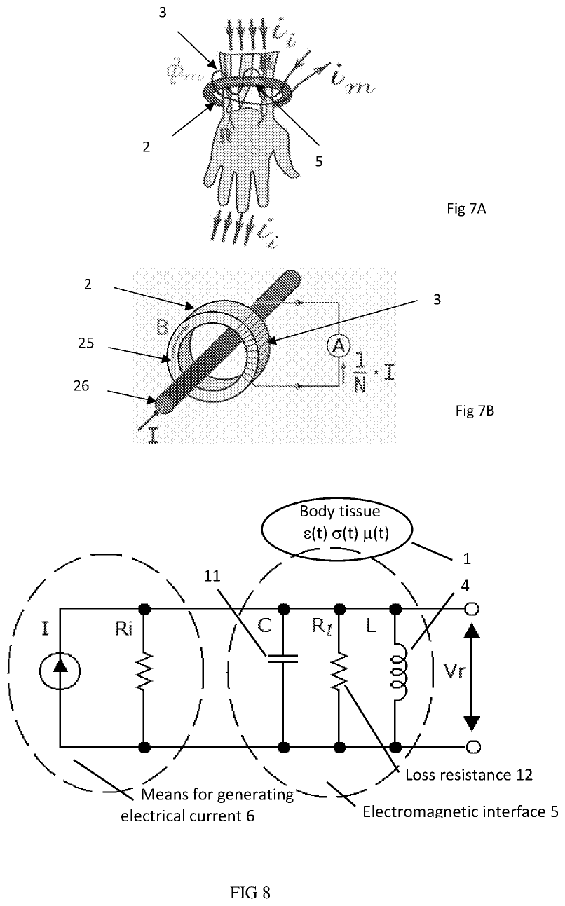

FIG. 7A shows the shape and direction of the magnetic field and the direction of the induced current when a toroidal core is placed on the wrist according to the invention. In the core 2, a magnetizing current iₘ passing through the winding 3 generates a magnetic flux 5, which induces an electric current iᵢ, the magnitude of which depends on the electrical impedance in the direction of the arm. The induced electric current iᵢ passes mainly through the blood vessels, both because of the directing of the magnetic field and because the electrical conductivity of the blood is several times higher than in the surrounding living tissues.

FIG. 7B shows the creation of a magnetic field in said toroidal core, and the current induced by it in a conductive material, e.g. in said blood vessel. The physical principle of electromagnetic induction follows Faraday's law. An electric current in the winding 3 with a number of turns N generates a magnetic flux 25 with a density B in the toroidal core which induces an electric current of N-times value through the opening of the toroidal core as arranged in an electrical conductor 26. The process is reversible, the same circuit is suitable for measuring the current through a toroidal core orifice, e.g. a current in a blood vessel. A current transformer is formed that can be used to measure the strength of an electric current induced in the body part, such as the current in the arm, flowing along a blood vessel.

FIG. 8 is an equivalent scheme for connecting the means for generating electrical current (6) with a body tissue via an electromagnetic interface (5), based on parallel resonant circuit containing the coil (4) with inductance L (wound around the strip 2) and a capacitance C (11). The capacitance C summarizes the variable capacitance introduced by the body (1) tissue and parasitic capacitances existing between the coil (4) windings. Variation of informative parameters of the body tissue, electrical permittivity ε(t), electrical conductivity σ(t), and magnetic permeability μ(t) reflect the 10 work of cardiopulmonary system. The loss resistance Rl (12) defines the selectivity and bandwidth (Q-factor) for a parallel resonance of resonant LC-circuit.

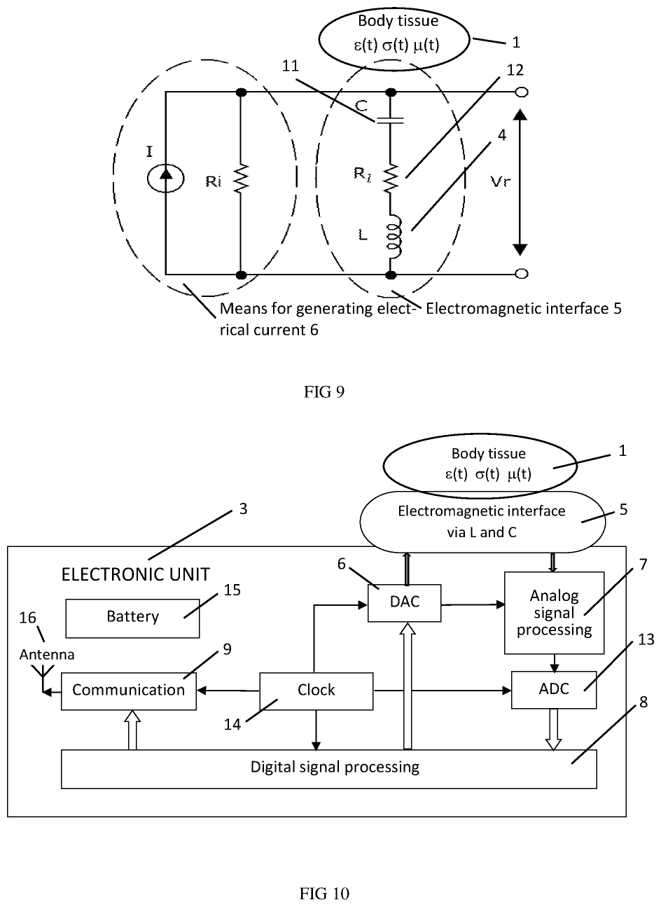

FIG. 9 is an equivalent scheme for connecting the means for generating electrical current 6 with body tissue 1 via the electromagnetic interface 5, based on serial resonant circuit containing the coil 4 with inductance L (wound around the strip) and a capacitance 11. The capacitance C summarizes the variable capacitance introduced by the body tissue and parasitic capacitances between the windings of coil 4. Variation of informative parameters of the body tissue, electrical permittivity ε(t), electrical conductivity σ(t), and magnetic permeability μ(t) reflect the work of cardiopulmonary system. The loss resistance Rl (12) defines the selectivity and bandwidth (Q-factor) for a serial resonance of resonant LC-circuit.

FIG. 10 shows an electronic unit 3 comprising the means for digital signal processing 8 connected with analog part of the bio-electromagnetic sensor through the means for generating electrical current 6 (based on a digital-to-analog converter DAC in FIG. 10) and an analog-to-digital converter ADC (14) to digitize and process the response signal 20 digitally after providing analog signal processing in 7. A non-galvanic electromagnetic interface 5 transforms the generated electrical current from 6 into an electromagnetic field applied to a vascularized body tissue 1. The body tissue 1 parameters as electrical permittivity ε(t), electrical conductivity σ(t), and magnetic permeability μ(t) reflect the work of cardiopulmonary system. The electrical response signal from the body tissue 1, coming through an electromagnetic interface 5, is amplified, filtered, detected and normalized in the means for analog signal processing 7 and digitized then by an analog-to-digital converter ADC (13). Informative part of the response signal from the interface 5 is extracted from its carrier component by demodulation, filtration and compensation using both analog signal processing in 7 and digital signal processing in 8: bridge circuits, compensation principles, hardware and digital modelling are taken into use for that. The work of all the components of electronic unit are synchronized by a master clock 14. A battery 15 based autonomous power supply is used. The means for digital data communications 9 is included for being in wireless connection with outer world (medical doctors, databases etc) via antenna 16.

FIG. 11 shows measurement of frequency response of impedance Z of a parallel LRC circuit, which describes the electromagnetic interface 5 connected to the body tissue 1 non-galvanically via inductance 4 of coil and capacitance 11.

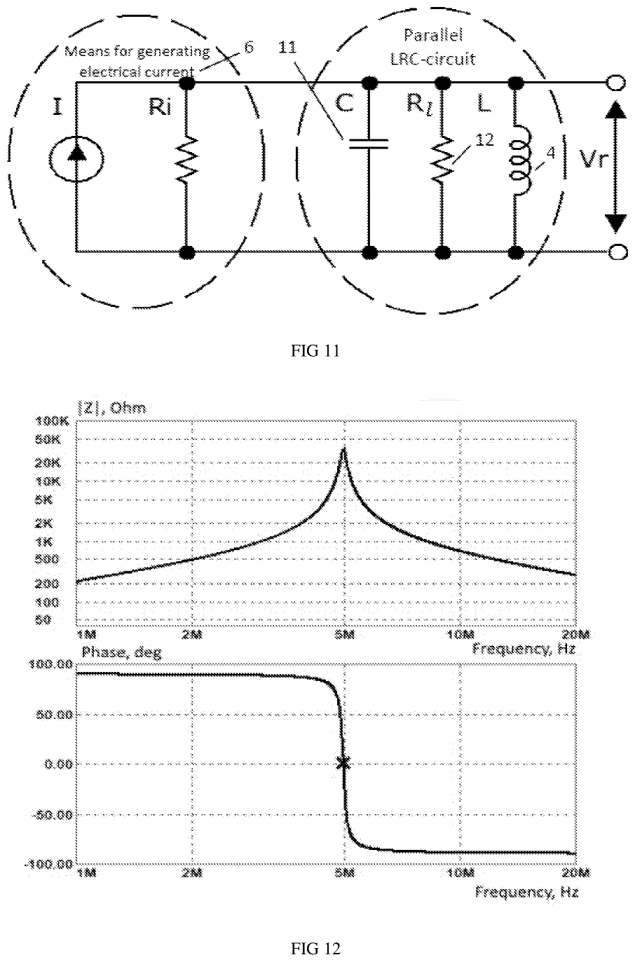

FIG. 12 shows measured frequency response of magnitude and phase of the impedance Z (body tissue 1 and electromagnetic interface 5) of the parallel resonant circuit given in FIG. 11 (the resonant frequency is 4.9 MHz).

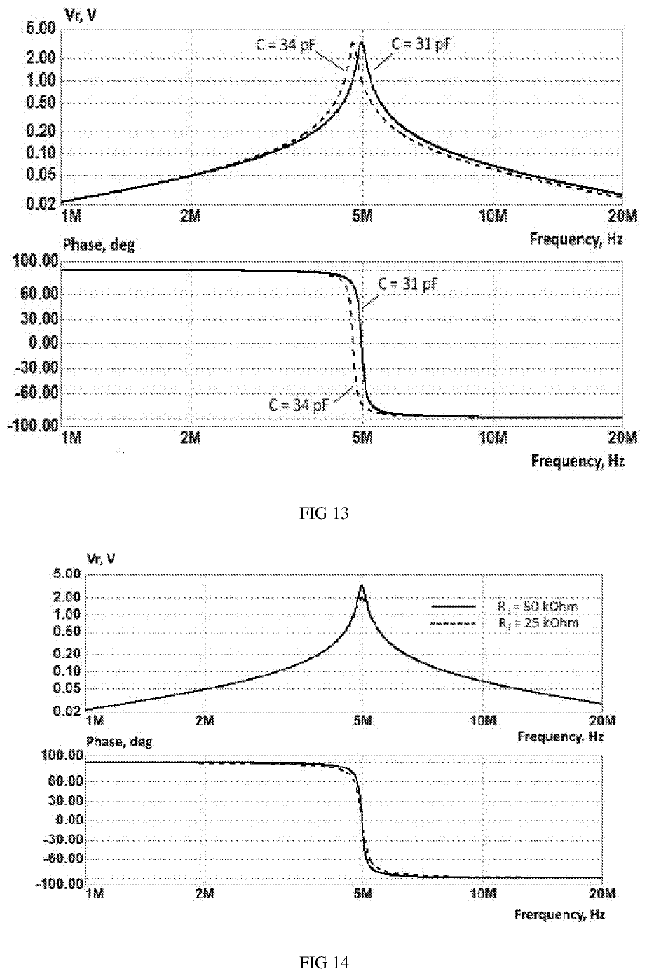

FIG. 13 shows the impact of the variation of capacitance C of the body tissue 1 from 31 to 34 pF (see also FIG. 11).

FIG. 14 shows the impact of the variations of losses in body tissue 1, when the loss resistance Rl (12) reduces from 50 to 25 kOhm (see also FIG. 11).

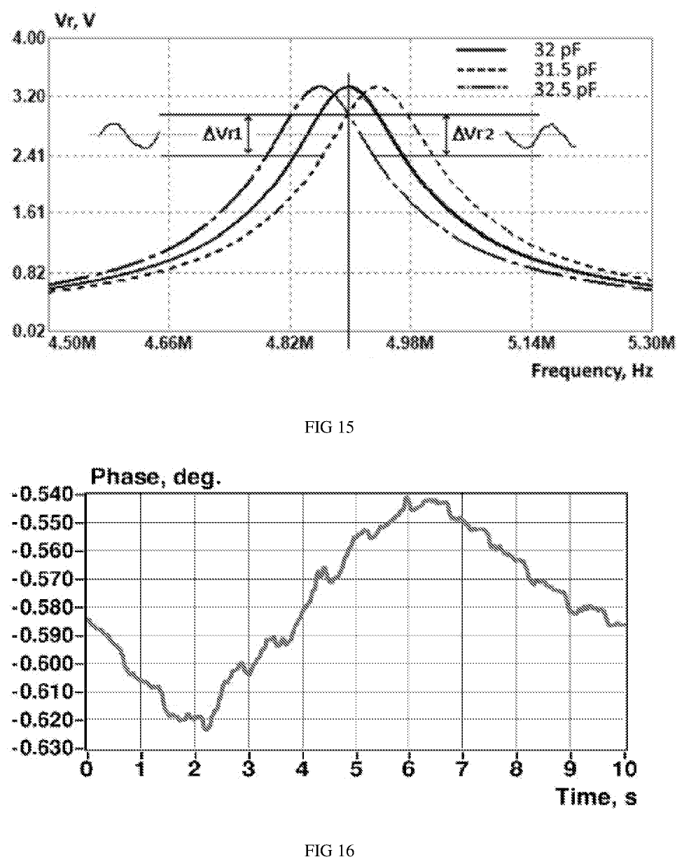

FIG. 15 shows the changes of the resonant frequency between 4.86 and 4.94 MHz of parallel RLC circuit.

FIG. 16 shows a graph of a measured impedance signal, phase modulated due to breathing.

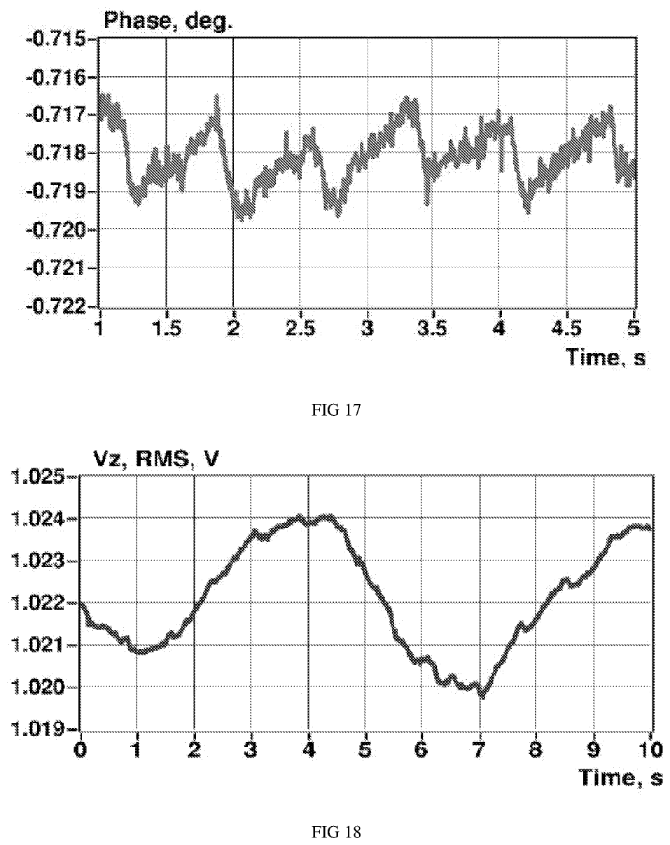

FIG. 17 shows a graph of measured impedance signal, phase modulated due to heart breathning.

FIG. 18 shows a graph of measured impedance signal, level modulated due to breathing.

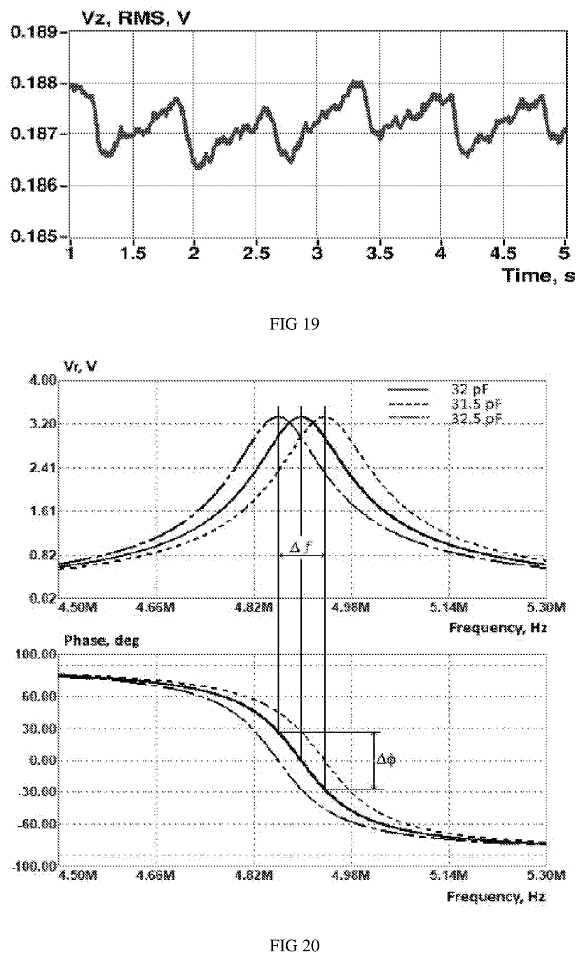

FIG. 19 shows a graph of measured impedance signal, level modulated due to heart beating. FIG. 20 shows changes of the resonant frequency Δf and phase Δφ of parallel RLC circuit (see FIG. 11) due to variation of the body tissue 1 capacitance 11 between 31.5 and 32.5 pF.

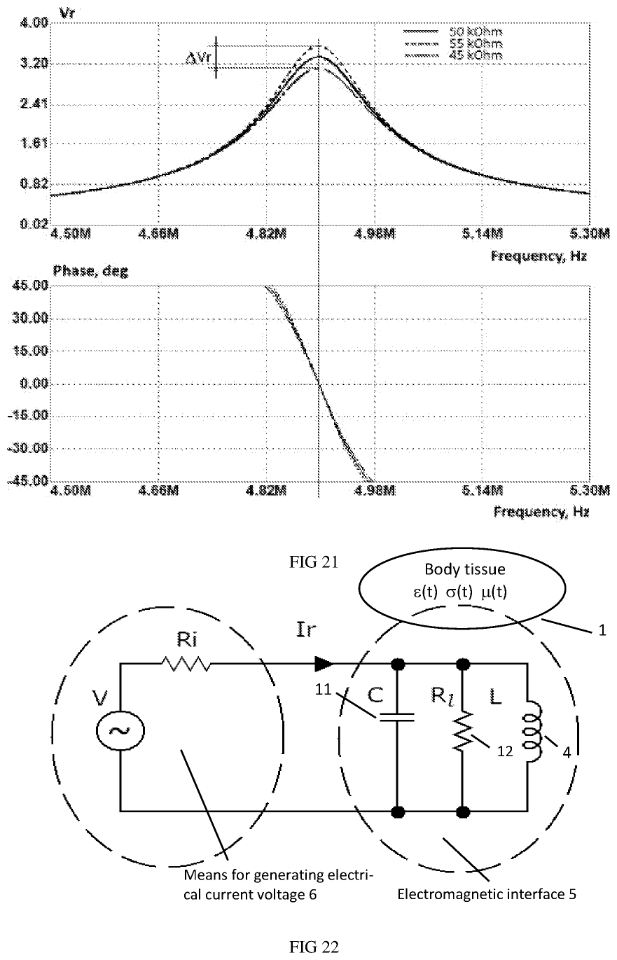

FIG. 21 shows changes of the level at resonant frequency of parallel RLC circuit (see FIG. 11) due to variation of losses in the body tissue 1, the loss resistance Rl (12) changes between 45 and 55 kOhm

FIG. 22 shows alternative measurement schemes when using the means for generating electrical current voltage V. A scheme for connecting the means for generating electrical current voltage V (6) with intrinsic resistance Ri to body tissue (1) via electromagnetic interface 5, based on parallel LRC circuit containing the coil 4 with inductance L (wound around a strip 2), a capacitance C (11), and a loss resistance Rl (12). Variation of informative parameters of the body tissue 1, as electrical permittivity ε(t), electrical conductivity σ(t), and magnetic permeability μ(t) reflect the work of cardiopulmonary system.

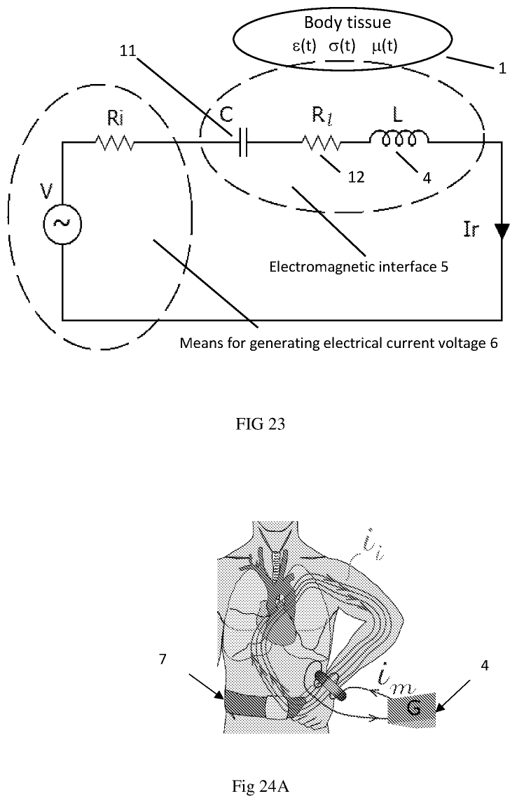

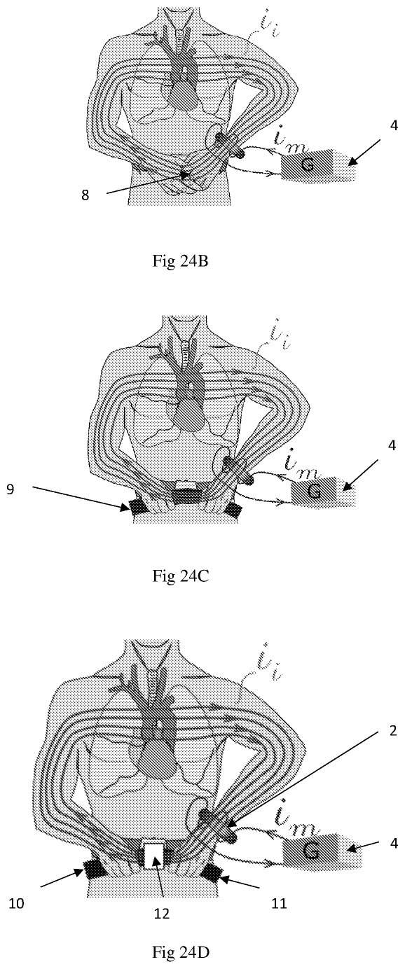

FIG. 23 shows a scheme for connecting the means for generating electrical current voltage (6) with intrinsic resistance Ri to body tissue (1) via electromagnetic interface (5), based on serial LRC resonant circuit containing the coil 4 with inductance L (wound around a strip 2), a capacitance C (11), and a loss resistance Rl (12). Variation of informative parameters of the body tissue 1, as electrical permittivity ε(t), electrical conductivity σ(t), and magnetic permeability μ(t) reflect the work of cardiopulmonary system.

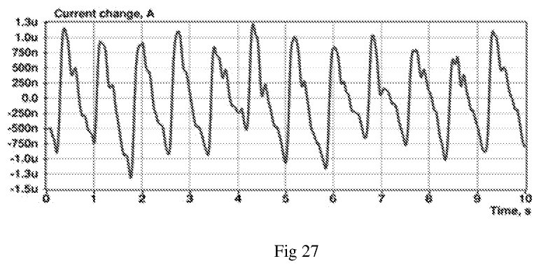

Electrical current can only flow in a closed circuit. Although the human bloodstream is a closed system through the arterial and venous blood vessels, it is difficult to induce a flow throughout the whole body. One solution is to artificially close the circulatory system in the section of interest, for example with additional electrodes, leaving the rest part out of effect, see FIGS. 26A and 26B. The additional electrodes are preferably superficial and non-invasive, for example via a galvanic or capacitive connection on the skin surface. FIG. 27 is a graph showing heart rate pulsation and its volume and nature as measured by the prototype device of Figures. 26A and 26B, respectively. The slow wave of the curve shows the effect of pulmonary respiration on heart rate. However, in principle, invasive electrodes can also be used, such as thin needle electrodes (micrometer-sized) inserted into the skin less than a millimeter deep. In some cases, it may be appropriate to use invasive techniques, in which the microelectrodes are inserted into a selected site in a blood vessel.

An alternative circuit closure is shown in FIG. 24A. The ring of the induced current iᵢ is closed through the belt 7 connecting the hand and the body through the electrical conductivity between the hand-strap-body and the electrical capacitance. In this case, a closed circuit is obtained in which the heart-lung and blood vessels are involved.

Another alternative way of closing the circuit is shown in FIG. 24B, where the circuit is closed through the electrical conductivity and capacitance between the closed hands 8.

A third alternative way of closing the circuit is shown in FIG. 24C, where the circuit is closed by means of an electrically conductive means 9 connecting both hands, such as a tube, bar, lever, handlebar or other electrically conductive material, e.g. sports equipment, e.g. as handlebars, handles for training and rehabilitation equipment, steering wheel for cars and other mobility equipment.

A fourth alternative circuit closure is shown in FIG. 24D, where the induced current circuit is closed by means of hands galvanically or capacitively connected to means 10 and 11 interconnected by a connecting device 12 through which a hand-to-hand connection is made to close the circuit. Closing is accomplished galvanically (by wire, cable, tape, braid or other electrically conductive means), capacitively (by a capacitor or other electrically capacitive structure) and magnetically (by a transformer or other inductively coupled structure) and by a high-frequency electromagnetic near-field, through a radio transceiver through air or other dielectric material as well as through optical coupling.

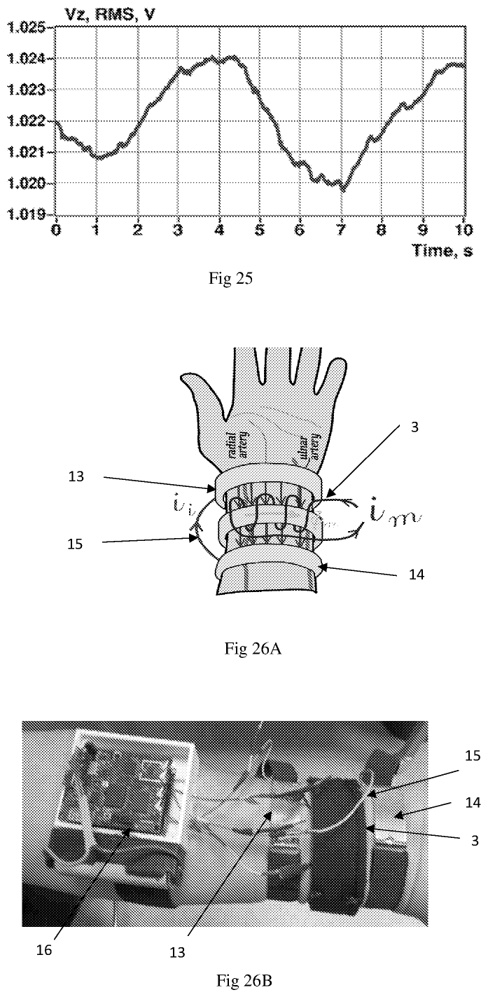

FIG. 25 is a lung respiration curve (a high amplitude but slow wave) with cardiac pulsation on it (with low amplitude, fast and jagged pulses). The curve is obtained from the applications shown in FIGS. 24A to 24D. The component corresponding to respiration prevails, but the amplitude of the component corresponding to the heart rate depends to a large extent on the specific solution (the largest in the case of FIG. 24D).

FIG. 26A shows a solution, in which the circuit is closed locally in the wrist by means of two additional electrodes 13 and 14 of conductive material, the induced current iᵢ closes through their electrical connection 15. The electrodes 13 and 14 have contacts with the body through galvanic conductivity and electrical capacitance between the electrodes and the body.

FIG. 26B is a photo of an example of the use, shown in FIG. 26A. A toroidal sensor coil 3 is attached to the wrist, which induces an electric current along the arm. Two gold electrodes 13 and 14 are added, between which the electrical wire connection 15 closes the circuit. By means of an electronic circuit 16 comprising a generator of alternating current signal and a detector, the volume and nature of the blood flow pulsation in the wrist section between the electrodes 14 and 15 can be measured.

Electrically connected electrodes can also be used to cut off the effects of certain anatomical parts from a closed circuit by shorting the electrodes mounted on them.

FIG. 27 shows a measured heart work curve with a slow change in amplitude due to respiration.

### ELEMENT LIST

Body tissue 1

### Strip 2

Electronic unit 3
Spiral winding (Coil) 4
Electromagnetic interface 5
Means for generating electrical current voltage (digital-to-analog converter DAC) 6
Means for analog signal processing 7
Means for digital signal processing 8
Means for digital data communications 9
Solenoidal winding 10

### Capacitance 11

Loss resistance 12
Analog-to-digital converter ADC 13
Master clock 14

### Battery 15

### Antenna 16

### REFERENCES

1. Jian Sun et al. (2018). An Experimental Study of Pulse Wave Measurements With Magnetic Induction Phase Shift Method, Tech Health Care, 2018; 26 (S1):157-167. doi:10.3233/THC-174526.2.

2. Jaan Ojarand, Siim Pille, Mart Min, Raul Land. Magnetic Induction Sensor for the Respiration Monitoring (2015), 10th International Conference in Bioelectromagnetism, 16-18 Jun. 2015 in Tallinn, Estonia.

3. Sharon Worcester (Apr. 6, 2020). Is Protocol-Driven COVID-19 Ventilation Doing More Harm Than Good? https://www.medscape.com/viewarticle/928236_print 1/2

### CLAUSES

1. A wearable bio-electromagnetic sensor comprising: an electronic unit, containing means for generating electrical current, means for analog signal processing, means for digital signal processing, and means for digital communications, and an electromagnetic interface for transforming said electrical current into an electromagnetic field applied to a body tissue.

2. The wearable bio-electromagnetic sensor according to clause 1, wherein said means for generating electrical current in said electronic unit applies a digital waveform synthesizer embedded into said means for digital signal processing by 15 converting said synthesized digital waveform into said electrical current by the aid of a digital-to-analog converter (DAC).

3. The wearable bio-electromagnetic sensor according to clause 1, wherein said electromagnetic interface for transforming said electrical current into an electromagnetic field exploits a magnetic component of the electromagnetic field applied to induce an electrical response in said body tissue.

4. The wearable bio-electromagnetic sensor according to clause 1, wherein said electromagnetic interface for transforming the electrical current into an electromagnetic field exploits an electric component of the electromagnetic field applied to induce said electrical response in said body tissue.

5. The wearable bio-electromagnetic sensor according to clause 1, wherein said electromagnetic interface for transforming the electrical current into an electromagnetic field, which exploits as magnetic component as well as the electric component of the electromagnetic field both applied to induce said electrical response in said body tissue.

6. The wearable bio-electromagnetic sensor according to clauses 1 and 3, wherein said electromagnetic interface for transforming said electrical current into an electromagnetic field applied to a body tissue comprises an inductive magnetic coil for inducing said electrical response in said body tissue.

7. The wearable bio-electromagnetic sensor according to clauses 1, 3 and 6, wherein said electromagnetic interface for transforming the electrical current into an electromagnetic field applied to a body tissue comprises said inductive magnetic coil and a capacitive component forming a resonant circuit for inducing said electrical response in said body tissue.

8. The wearable bio-electromagnetic sensor according to clauses 1 and clauses 4, wherein said electromagnetic interface for transforming the electrical current into an electromagnetic field applied to a body tissue comprises capacitive electrodes for inducing said electrical response in said body tissue.

9. The wearable bio-electromagnetic sensor according to f clauses 1, 4 and 8, wherein said electromagnetic interface for transforming the electrical current into an electromagnetic field applied to a body tissue comprises said capacitive electrodes and an inductive component to form a resonant circuit for inducing said electrical response in said body tissue.

10. The wearable bio-electromagnetic sensor according to clauses 1 and 5, wherein said electromagnetic interface for transforming the electrical current into an electromagnetic field applied to a body tissue comprises said inductive magnetic coil and said capacitive electrodes forming a resonant circuit for inducing said electrical response in said body tissue.

11. The wearable bio-electromagnetic sensor according to clauses 1, 6, 7 and 10, wherein said inductive magnetic coil is wound as a spiral winding on a circular core.

12. The wearable bio-electromagnetic sensor according to clauses 1, 6, 7, and 10, wherein said inductive magnetic coil is wound as a circular winding on a circular core.

13. The wearable bio-electromagnetic sensor according to anyone of clauses 1, 11 and 12, wherein said circular core is a closed loop of magnetic material.

14. The wearable bio-electromagnetic sensor according to clauses 1, 11, 12 and 13, wherein said circular core is a closed loop of magnetic material having one or more discontinuities as a gaps of air and other non-magnetic materials.

15. The wearable bio-electromagnetic sensor according to clauses 1, 11 and 12, wherein said circular core is a loop of non-magnetic material.

16. The wearable bio-electromagnetic sensor according to clauses 1, 11, 12, 13, 14, and 15, wherein a form of said circular core is modified to fit to round shape body parts on which said wearable bio-electromagnetic sensor is placed

17. The wearable bio-electromagnetic sensor according to clauses 1, 8, 9 and 10, wherein said capacitive electrodes have a circular shape.

18. The wearable bio-electromagnetic sensor according to clauses 1, 8, 9, and 10, wherein said capacitive electrodes have a semi-circular shape.

19. The wearable bio-electromagnetic sensor according to clauses 1 and 11, wherein said capacitive electrodes have a circular form with discontinuities modified to fit to round shape body parts on which said wearable bio-electromagnetic sensor is placed.

20. The wearable bio-electromagnetic sensor according to clauses 1, 3, 4, and 5, wherein said means for processing analog signals in electronic unit contains a detector of variations in electrical response to said electromagnetic field applied to said body tissue.

21. The wearable bio-electromagnetic sensor according to clauses 1, 3, 4, and 5, wherein said means for processing analog signals in electronic unit contains a detector of level variations in said electrical response to said electromagnetic field 5 applied to said body tissue.

22. The wearable bio-electromagnetic sensor according to clauses 1, 3, 4 and 25, wherein said means for processing analog signals in electronic unit contains a synchronous detector of level variations in electrical response to said electromagnetic field applied to said body tissue.

23. A work of said synchronous detector of level variations in clause 22 is controlled synchronously with a frequency of said electromagnetic field applied to said body tissue.

24. A work of said detector of level variations in clauses 21 and 22, operates at a frequency, detuned 0.1 to 10% from said resonant frequency of electromagnetic interface for transforming the electrical current into an electromagnetic field applied to a body tissue.

25. The wearable bio-electromagnetic sensor according to clauses 1, 21, 22 and 23, wherein said detectors of level variations in said electrical response to said electromagnetic field applied to said body tissue, in which said variations express electrical energy losses due to variations of electrical conductivity σ(t).

26. The wearable bio-electromagnetic sensor according to clauses 21, 22, and 23, wherein said level variations in said electrical response to said electromagnetic field applied to said body tissue express electrical energy losses due to variations of electrical conductivity σ(t) are caused by pulsation of blood amount and 30 pressure in said body tissue accordingly to heart beating.

27. The wearable bio-electromagnetic sensor according to clauses 1, 3, 4, and 5, wherein said means for processing analog signals in electronic unit contains a detector of phase shift variations between said electric response and said electromagnetic field applied to said body tissue.

28. The wearable bio-electromagnetic sensor according to clauses 1, 3, 4 and 5, wherein said means for processing analog signals in electronic unit contains a detector of real and imaginary parts of complex variations between said electric response and said electromagnetic field applied to said body tissue.

29. The wearable bio-electromagnetic sensor according to clauses 1, 7, 9 and 10, 10 wherein said means for processing analog signals in said electronic unit contains a detector of resonant frequency variations of said resonant circuit in said electromagnetic interface for transforming the electrical current into an electromagnetic field applied to a body tissue.

30. The wearable bio-electromagnetic sensor according to clauses 1, 7, 9, 10 and 29, wherein said means for processing analog signals in said electronic unit contains a detector of phase shift due to resonant frequency variations of said resonant circuit in said electromagnetic interface for transforming the electrical current into an electromagnetic field applied to a body tissue.

31. The wearable bio-electromagnetic sensor according to clauses 1, 26, 27, 28 and 29, wherein said phase and frequency and real and imaginary parts variations in said electrical response to said electromagnetic field applied to said body tissue express variations of electrical permittivity electrical permittivity ε(t) due to oxygenation of said body tissue accordingly to breathing of lungs.

32. The wearable bio-electromagnetic sensor according to clauses 1 and 19, wherein said means for processing analog signals in electronic unit contains a detector of variations in electrical response to said electromagnetic field applied to said body tissue express variations of magnetic permeability μ(t) accordingly to blood flow.

33. The wearable bio-electromagnetic sensor according to clause 1, wherein said means for processing analog signals includes a compensator of a permanent part (carrier component) of said electric response to said electromagnetic field applied to body tissue.

34. The wearable bio-electromagnetic sensor according to clause 1, wherein said means for processing analog signals includes a bridge circuit for minimizing (zero immersion) said permanent part of said electric response to said electromagnetic field applied to body tissue.

35. The wearable bio-electromagnetic sensor according to clause 1, an analog output of said means for processing analog signals in said electronic unit is converted into a digital input of said means for digital signal processing by an analog-to-digital converter (ADC).

36. The wearable bio-electromagnetic sensor according to 1, in which said means for digital signal processing in said electronic unit provides a post-processing of digitized output of said means for processing analog signals performing filtering, linearization, post-detection, error minimization, uncertainty reduction, extraction of essential parameters and other required mathematical and logical operations.

37. The wearable bio-electromagnetic sensor according to clauses 1 and 2, in which said means for digital signal processing in said electronic unit provides said digital synthesis of said digital waveform for the converting it into said electrical current by the aid of said digital-to-analog converter (DAC).

38. The wearable bio-electromagnetic sensor according to clause 1, in which said means for digital signal processing in said electronic unit provides a coding of said extracted essential parameters into a suitable format for a means for digital data communications via included transceiver and antenna.

39. The wearable bio-electromagnetic sensor according to clause 1, in which said electronic unit comprises a master clock, which synchronizes the work of said components in it.

40. A device for determining physiological parameters of a body organ in a convex body, the device comprising a toroidal core electromagnet shaped to follow the convex surface of the body ⅛ to 1/1 of the convex surface, an alternating current generator, and a means for measuring and processing a response signal.

41. The device of clause, wherein the core of the toroidal core electromagnet is a helically wound coil connected to an alternating current generator.

42. The device according to clauses 40 to 41, comprising means for closing the path of current induced by an electromagnet and passing through the body.

43. The device according to clause 42, wherein the means is an electrically conductive component connecting the two hands.

44. The device of clause 43, wherein the device is a metal object.

45. The device of clauses 40 to 42, wherein the device is an electrically conductive component connecting the arm and the body.

46. The device of clauses 42 to 45, wherein the device is a belt around the body or round body portion.

47. The device of clauses 40 to 46, wherein the means for measuring and processing the response signal comprises a current transformer for measuring the response signal.

48. The device of clauses 40 to 47, comprising electrodes for measuring the response signal.

49. The device of clauses 40 to 47, comprising an ammeter for measuring a response signal in the form of an electric current.

50. The device of clauses 40 to 48, comprising a voltmeter for measuring a response signal in the form of an electrical voltage.

51. The device of clauses 40 to 49, wherein the means for measuring and processing the response signal comprises an electronic device for obtaining physiological parameters from the results of measuring the induced current and the response signal.

1-39. (canceled)

40. A method for determining physiological parameters, the method comprising the steps of:
placing an electromagnet with a toroidal core, having a transversely wound winding on said toroidal core, on a convex body part so that the shape of the toroidal core follows ⅛ to 1/1 extent the convex surface of the body part,
inducing an alternating electric current in said body organ,
galvanically or electromagnetically receiving a response signal from said body organ, and determining said physiological parameters of the body from said response signal.

41. The method according to claim 40, wherein said body organ is a blood vessel and lung function parameters are determined from the response signal.

42. The method according to 40, wherein said body organ is a blood vessel and heart function parameters are determined from the response signal.

43. The method according to claims 40, wherein said body organ is a blood vessel and vascular function is determined from the response signal.

44. The method according to claim 40, wherein two capacitive or galvanic electrodes are placed on each side of the toroidal core of said electromagnet and are connected to close an intracorporal circuit path.

45. The method according to claim 44, wherein said two electrically connected electrodes are used to disconnect certain anatomical parts from the intracorporeal circuit path by shorting the electrodes.

46. The method according to claim 45, wherein the two electrodes are electrically connected to each other via a short-circuit ammeter or an electronic circuit operating equivalent thereto such as a current-voltage converter, for measuring the current of the response signal.

47. The method according to claim 46, wherein the current of the response signal is measured by a toroidal core current transformer.

48. The method according to claim 45, wherein belts arranged around the body are used to close the intracorporal circuit pat

49. The method according to claim 45, wherein the intracorporal circuit is closed through an electrically conductive device.

50. The method according to claim 49, wherein said electrically conductive device is selecting from the group consisting of a sports aid, ski poles, walking poles, a bicycle handlebar, a motorcycle handlebar, a handle for a training equipment or a rehabilitation equipment, and a steering wheel for a vehicle.

51. The method according to claim 49, wherein the intracorporeal circuit path is closed through an electrically conductive device integrated into a garment.

52. The method according to claim 40, wherein the circuit path is closed by a connecting device through which the connection between the hands is made capacitively, magnetically, optically or via a near electromagnetic field.

53. A sensor device for determining physiological parameters of an individual, the device comprising:
a toroidal magnetic coil, comprising a circular core with a spiral primary winding wound around it, wherein said circular core is adapted to be placed around a convex shaped body part;
an electronic unit, comprising
means for generating an electrical input current into said spiral primary winding, thereby generating an electromagnetic field in said convex shaped body part, said electromagnetic field being in the direction of a body organ located inside said convex shaped body part and thereby generating corresponding current in said body organ;
means for receiving a response signal from said convex shaped body part;
means for calculating physiological parameters from said response signal and said input current, said physiological parameters selected from a group consisting of lung function parameters, heart function parameters and vascular function parameters.

54. The sensor device as in claim 53, comprising a first electrode to be placed on said convex shaped body part on first side of the circular core and a second electrode to be placed said convex shaped body part on opposite side of the circular core, wherein said first electrode and said second electrode are connected with each other through a wire.

55. The sensor device as in claim 54, wherein said first electrode and said second electrode are non-invasive electrodes.

56. The sensor device as in claim 55, wherein said first electrode and said second electrode are capacitive electrodes.

57. The sensor device as in claim 52, comprising a solenoidal secondary winding wound along said circular core, and means for generating a second electrical input into said solenoidal secondary winding.

58. The sensor device as in claim 52, wherein at least part of the circular core is made of a flexible magnetic material, wherein said magnetic material is 1/10 to ½ of the full extent of the circular core.

59. The sensor device as in claim 52, comprising means for connecting an intracorporal circuit, said means selected from a group consisting of a sports aid, ski poles, walking poles, a bicycle handlebar, a motorcycle handlebar, a handle for a training equipment or a handle for a-rehabilitation equipment, a steering wheel for a vehicle, a belt and an electrically conductive material integrated into a garment.
